(19) United States
(12) Patent Application Publication
(10) Pub. No.: US 2007/0065494 A1
(43) Pub. Date: Mar. 22, 2007

(54) Formulations and Methods for Enhancing the Transdermal Penetration of a Drug

(75) Inventors: Angela N. Anigbogu, Salt Lake City, UT (US); Samir Roy, Salt Lake City, UT (US); Vesna Anteljevic, Salt Lake City, UT (US)

Correspondence Address: THORPE NORTH & WESTERN, P.O. BOX 1219, SANDY, UT, 84091-1219, US

(73) Assignee: WATSON LABORATORIES, INC., Salt Lake City, UT (US)

(21) Appl. No.: 11/461,377

(22) Filed: Jul. 31, 2006

Related U.S. Application Data
(60) Provisional application No. 60/705,289, filed on Aug. 3, 2005.

(51) Int. Cl. A61K 9/70; A61K 31/22

(52) U.S. Cl. 424/449; 514/546

(57) ABSTRACT

Methods and formulations of enhancing the permeability the skin of a subject to a drug are disclosed. The method may include administering a combination of lauryl alcohol and isopropyl myristate as a penetration enhancer to the area of skin to provide synergistically enhanced penetration of the drug.

### PRIORITY DATA

The present application claims the benefit of U.S. Provisional Patent Application Ser. No. 60/705,289, filed Aug. 3, 2005, which is incorporated herein by reference.

### FIELD OF THE INVENTION

The present invention relates to methods and formulations that synergistically enhance the penetration of a drug through the skin of a subject. Accordingly, this invention involves the fields of chemistry, pharmaceutical sciences, medicine and other health sciences.

### BACKGROUND OF THE INVENTION

Transdermal delivery of drugs is a well established route of administration that typically provides many advantages over other routes such as oral and parenteral delivery. Recognized advantages of transdermal drug delivery include greater patient comfort and convenience, reduced dosing frequency, elimination of hepatic first pass metabolism, and a high degree of control over drug plasma concentrations. All of these advantages lead to greater patient compliance and allow greater freedom in designing effective pharmacotherapy regimens.

Despite such advantages, transdermal drug formulations typically face a number of challenges. Skin irritation, slow onset of action times, instability during storage, and the amount of skin area required to deliver a therapeutically effective amount of the drug through the skin and into the serum are issues that typically hamper most transdermal drug delivery efforts.

Chemical skin penetration enhancers are well know for use in transdermal drug delivery in order to address the main challenge recited above, namely, increasing the rate at which the drug passes through the skin and into the serum. Accelerated penetration of the drug provides a number of advantages such as decreasing the time required to attain therapeutic drug serum levels and the ability to reduce the size of skin area over which the drug must be administered. Unfortunately, the inclusion of skin penetration enhancers also causes, or at least contributes to a number of the other challenges recited above, such as reacting with the drug in the transdermal formulation, contributing to or causing skin irritation, and affecting adhesiveness of transdermal patches.

A number of specific compounds have been investigated as transdermal penetration enhancers. Examples of agents used as enhancers may be found in U.S. Pat. Nos. 5,601,839; 5,006,342; 4,973,468, 4,820,720; 4,006,218, 3,551,154; and 3,472,931. The effectiveness of a penetration enhancer is extremely important because the more potent the enhancer, the less amount is required to achieve an enhancing effect. Of course, when less enhancer is required, the challenges of successfully including the enhancer in the transdermal formulation are reduced.

As a result, penetration enhancers and penetration enhancer compositions that are highly potent for a broad spectrum of drugs continue to be sought.

### SUMMARY OF THE INVENTION

Accordingly, the present invention provides methods and formulations that enhance penetration of a drug through the skin of a subject. In one aspect, such a method may include enhancing penetration of a drug through an area of skin by administering a combination of lauryl alcohol and isopropyl myristate as a penetration enhancer to the area of skin to provide synergistically enhanced penetration of the drug.

The synergistic effect of the combination of lauryl alcohol and isopropyl myristate can cause penetration enhancement of a drug that is greater than the additive effect of an equivalent volume of each individual enhancer present in the formulation. As such, in one aspect, the transdermal penetration enhancement can be from about 5% to about 150% greater than would be expected of an additive effect from using lauryl alcohol and isopropyl myristate. In another aspect, the penetration enhancement is from about 5% to about 75% greater than would be expected. In yet another aspect, the penetration enhancement is from about 10% to about 50% greater than would be expected.

The ratio of lauryl alcohol to isopropyl myristate can vary depending on the desired drug enhancement characteristics of the formulation, the characteristics of the drug, particular adhesives used, etc. In one aspect, the penetration enhancer may include lauryl alcohol and isopropyl myristate in a ratio of from about 1:5 to about 5:1. In another aspect, the penetration enhancer may include lauryl alcohol and isopropyl myristate in a ratio of from about 1:4 to about 4:1. In another aspect, the penetration enhancer may include lauryl alcohol and isopropyl myristate in a ratio of from about 1:3 to about 3:1. In yet another aspect, the penetration enhancer may include lauryl alcohol and isopropyl myristate in a ratio of from about 1:2 to about 2:1. In yet another aspect, the penetration enhancer may include lauryl alcohol and isopropyl myristate in a ratio of about 1:1. In a further aspect, the penetration enhancer may include lauryl alcohol and isopropyl myristate in a ratio of about 4:1.

The permeation enhancer of the present invention may increase the permeability of the skin of a subject to a variety of drugs. One class of drugs that may benefit from the permeation enhancer combination is hormones. Any hormone known to one skilled in the art is considered to be within the scope of the present invention, however sex hormones are of particular interest. Specific examples of sex hormones include, without limitation, testosterone, norethindrone, norethindrone acetate, estradiol, norelgestromin, and mixtures, salts, isomers, or analogues thereof. Testosterone is a sex hormone for which the combination of lauryl alcohol and isopropyl myristate shows particular merit.

The lauryl alcohol can be administered to the subject in any order with respect to the isopropyl myristate. Thus, the lauryl alcohol and the isopropyl myristate can be administered separately, or they can be administered as a single enhancer composition. As such, the lauryl alcohol can be administered either prior to, concurrently with, or following the isopropyl myristate. In one aspect of the present invention, the lauryl alcohol is administered concurrently with the isopropyl myristate. Similarly, the combination of lauryl alcohol and isopropyl myristate can be administered either prior to, concurrently with, or following the drug. In one aspect, the combination of lauryl alcohol and isopropyl myristate is administered concurrently with the drug. In another aspect, the combination of lauryl alcohol and isopropyl myristate can be administered before, during, and after the drug. In yet another aspect, the combination of lauryl alcohol and isopropyl myristate can be administered as a single enhancer composition.

In another aspect of the present invention, a penetration enhancer composition for use as recited herein is also provided. Such a penetration enhancer may include a combination of lauryl alcohol and isopropyl myristate, and may provide synergistically enhanced penetration of a drug through the skin.

The present invention additionally encompasses transdermal formulations which can be administered according to the present methods. In one aspect, such a formulation can include a pharmaceutically acceptable carrier, a therapeutically effective amount of a drug, and a penetration enhancer composition as recited herein.

While the amount of lauryl alcohol included in the formulation can vary depending on a number of criteria, such as the particular drug to be delivered, the chemical makeup of the carrier, etc., in one aspect the lauryl alcohol and the isopropyl myristate can each be from about 0.5% w/w to about 20% w/w of the transdermal formulation. In another aspect, the lauryl alcohol and the isopropyl myristate can each be from about 1% w/w to about 10% w/w of the transdermal formulation. In another aspect, the lauryl alcohol and the isopropyl myristate can each be from about 2.5% w/w to about 7.5% w/w of the transdermal formulation. In yet another aspect, the lauryl alcohol and the isopropyl myristate can each be about 5% w/w of the transdermal formulation. In a further aspect, the lauryl alcohol and the isopropyl myristate can each be less than or equal to about 5% w/w of the transdermal formulation.

The pharmaceutically acceptable carriers of the present invention can be any carrier known to one skilled in the art. In one aspect, the pharmaceutically acceptable carrier can be a biocompatible polymer. Biocompatible polymers can include, without limitation, rubbers; silicone polymers and copolymers; acrylic polymers and copolymers; and mixtures thereof. In one aspect, the biocompatible polymer is a rubber, which can be any useful rubber known to one skilled in the art, including natural and synthetic rubbers; plasticized styrene-rubber block copolymers, etc., and mixtures thereof. In another aspect, the biocompatible polymer can include silicone polymers, polysiloxanes, and mixtures thereof. In yet another embodiment, the biocompatible polymer can include acrylic polymers, polyacrylates, and mixtures thereof. In a further embodiment, the biocompatible polymer can include vinyl acetates, ethylene-vinyl acetate copolymers, polyurethanes, plasticized polyether block amide copolymers, and mixtures thereof.

The present invention also contemplates liquid reservoir system (LRS) formulations. In one aspect, the pharmaceutically acceptable carrier can include a viscous material suitable for inclusion in a liquid reservoir. One example of a viscous material can include, without limitation, a material that forms a gel.

The transdermal formulations of the present invention may take numerous specific embodiments. In one aspect, the formulation may be a transdermal patch. Transdermal patches may include any type of patch known to one skilled in the art, including transdermal matrix patches, liquid reservoir patches, etc. In another aspect, the transdermal formulation can be a topical formulation. Topical formulations can include, without limitation, creams, lotions, ointments, gels, pastes, mousses, aerosols, sprays, waxes, balms, suppositories, and mixtures or combinations thereof. Any one of a number of specific ingredients may be used in order to provide a specifically desired transdermal formulation, such as diluents, excipients, emollients, plasticizers, solubilizers, skin irritation reducing agents, stabilizing compounds, or mixtures thereof.

In another aspect, a method of transdermally delivering a drug with enhanced penetration through an area of skin on a subject is provided. Such a method typically includes administering a transdermal formulation as recited herein to the area of the subject's skin.

The present invention also contemplates a kit for administering a transdermal formulation having a drug with enhanced penetration through an area of skin on a subject. In one aspect, the kit may include a pharmaceutically acceptable carrier having a therapeutically effective amount of a drug admixed therein, an effective amount of lauryl alcohol, and an effective amount of isopropyl myristate. The lauryl alcohol and the isopropyl myristate may provide synergistically enhanced transdermal penetration of the drug. The kit may also include instructions describing a method of using the transdermal formulation. In one aspect, the lauryl alcohol and the isopropyl myristate may be combined as a single penetration enhancer composition. In another aspect, they may be separately contained within the kit.

In a specific embodiment of the present invention, a method of transdermally delivering a drug with enhanced penetration through an area of skin on a subject is provided. The method can include administering to the area of skin, a transdermal formulation including a pressure sensitive acrylic polymer adhesive of from about 55% w/w to about 85% w/w of the transdermal formulation, testosterone of from about 5% w/w to about 12% w/w of the transdermal formulation, polyvinylpyrrolidone of from about 8% w/w to about 12% w/w of the transdermal formulation, and a penetration enhancer composition including a combination of from about 2% w/w to about 8% w/w of lauryl alcohol and from about 2% w/w to about 8% w/w of isopropyl myristate. The penetration enhancer composition may provide synergistically enhanced penetration of a drug through an area of skin of from about 10% to about 50% greater than would be expected of an additive effect from using lauryl alcohol and isopropyl myristate.

In another specific embodiment, a transdermal formulation having enhanced penetration of a drug is provided. The transdermal formulation may include a pressure sensitive acrylic polymer adhesive of from about 55% w/w to about 85% w/w of the transdermal formulation, testosterone of from about 5% w/w to about 12% w/w of the transdermal formulation, a solubilizer of from about 8% w/w to about 12% w/w of the transdermal formulation, and a penetration enhancer composition including lauryl alcohol of from about 2% w/w to about 8% w/w of the transdermal formulation and isopropyl myristate of from about 2% w/w to about 8% w/w of the transdermal formulation. The penetration enhancer composition may provide synergistically enhanced penetration of the testosterone through an area of skin of a subject.

Definitions of Terms

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “an excipient” includes reference to one or more of such excipients.

As used herein in, “lauryl alcohol” or “1-Dodecanol” refers to a compound having the general chemical structure: 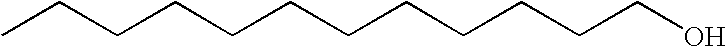
Lauryl alcohol is well known in the art, and is listed as monograph 3439 on pg. 3443 of the Merck Index (13^{th} ed. 2001), which is incorporated herein by reference.

As used herein, “isopropyl myristate” or “IPM” refers to a compound having the general chemical structure: 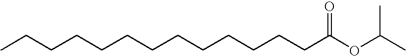
IPM is well known in the art, and is listed as monograph 5235 on pg. 5238 of the Merck Index (13^{th} ed. 2001), which is incorporated herein by reference.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, the terms “formulation” and “composition” are used interchangeably and refer to a mixture of two or more compounds, elements, or molecules. In some aspects the terms “formulation” and “composition may be used to refer to a mixture of one or more active agents with a carrier or other excipients. The terms “drug,” “pharmaceutical,” “active agent,” and “bioactive agent” are also used interchangeably to refer to a pharmacologically active substance or composition. These terms of art are well-known in the pharmaceutical and medicinal arts.

As used herein, “transdermal” refers to the route of administration taken by a drug that is applied to and absorbed through an area of skin. In some aspects, the skin may be substantially unbroken. Thus the terms “transdermal formulation” and “transdermal composition” can be used interchangeably, and refer to formulations or compositions that are applied to a surface of the skin and transdermally absorbed. Examples of transdermal formulations include but are not limited to, ointments, creams, gels, transdermal patches, sprays, lotions, mousses, aerosols, nasal sprays, buccal and sublingual tablets and tapes, vaginal rings, and pastes. The term “transdermal administration” thus refers to the transdermal application of a formulation or composition. Transdermal administration can be accomplished by applying, pasting, rolling, attaching, pouring, pressing, rubbing, etc., of a transdermal preparation or formulation onto a skin or mucosal surface. These and additional methods of administration are well-known in the art.

The terms “transdermal delivery system,” “transdermal patches” or simply “patches” refer to a matrix or liquid reservoir type of transdermal delivery device which is used to transdermally deliver defined doses of a substance, over a specific application period.

By the term “matrix”, “matrix system”, or “matrix patch” is meant a composition comprising an effective amount of a drug dissolved or dispersed in a polymeric phase, often a pressure sensitive adhesive, which may also contain other ingredients, such as a permeation enhancers, skin irritation reducing agents, excipients, plasticizers, solubilizers, emollients, and other optional ingredients. This definition is meant to include embodiments wherein such polymeric phase is laminated to a pressure sensitive adhesive or used within an overlay adhesive.

The general structure of a matrix-type patch is known to those skilled in the art. Such structure typically includes a drug-impermeable occlusive backing laminated to the distal side of a solid or semisolid matrix layer comprised of a homogeneous blend of the drug, a polymeric pressure sensitive adhesive carrier, and optionally one or more skin permeation enhancers, and a temporary peelable release liner adhered to the proximal side of the matrix layer. In use, the release liner is removed prior to application of the patch to the skin. Matrix patches are known in the art of transdermal drug delivery. Examples without limitation, of adhesive matrix transdermal patches are those described or referred to in U.S. Pat. Nos. 5,985,317, 5,783,208, 5,626,866, 5,227,169, 5,122,383 and 5,460,820 which are incorporated by reference in their entirety.

Additionally, the general structure of a liquid reservoir system (LRS) type patch is also known. Such patches typically comprise a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. The drug and any penetration enhancers are contained in the fluid in desired amounts. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety.

The terms “skin,” “skin surface,” “derma,” “epidermis,” and similar terms are used interchangeably herein, and refer to not only the outer skin of a subject comprising the epidermis, but also to mucosal surfaces to which a drug composition may be administered. Examples of mucosal surfaces include the mucosal of the respiratory (including nasal and pulmonary), oral (mouth and buccal), vaginal, introital, labial, and rectal surfaces. Hence the terms “transdermal” encompasses “transmucosal” as well.

As used herein, “enhancement,” “penetration enhancement,” or “permeation enhancement,” refer to an increase in the permeability of the skin to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which is incorporated by reference herein.

The terms “synergism,” “synergistic,” and “synergistically” refer to a phenomenon in which the combined effect of at least two active components of a composition or mixture is more than additive. In other words, the effect attained by the combination of components is greater than the effect of each component measured by itself and then added together. The determination of synergism is well known to those of ordinary skill in the art. For the case in which both agents have some efficacy individually, the expected effect of a combination can be measured in some aspects, by using Loewe Additivity values (W. R. Greco et al., Pharmacological Reviews 47:331-385 (1995)).

As used herein, “effective amount” of an enhancer refers to an amount sufficient to increase the penetration of a drug through the skin to a selected degree. Methods for assaying the characteristics of permeation enhancers are well-known in the art. See, for example, Merritt et al., “Diffusion Apparatus for Skin Penetration,” J. of Controlled Release 61 (1984), incorporated herein by reference in its entirety. Thus, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier,” and “carrier” may be used interchangeably, and refer to any inert and pharmaceutically acceptable material that has substantially no biological activity, and makes up a substantial part of the formulation. The carrier may be polymeric, such as an adhesive, or non-polymeric and is generally admixed with other components of the composition (e.g., drug, binders, fillers, penetration enhancers, anti-irritants, emollients, lubricants, etc., as needed) to comprise the formulation.

The term “admixed” means that the drug and/or enhancer can be dissolved, dispersed, or suspended in the carrier. In some cases, the drug may be uniformly admixed in the carrier.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

### DETAILED DESCRIPTION OF THE INVENTION

Applicants have discovered that the combination of lauryl alcohol and isopropyl myristate provides synergistic penetration enhancement of a drug through an area of skin. In addition to enhancing the penetration of various drugs, the combination of lauryl alcohol and isopropyl myristate allow the use of lower concentrations of each enhancer, thus reducing levels of skin irritation often associated with higher enhancer concentrations. Therefore, the present invention involves methods and formulations for synergistically enhancing the penetration of a drug through the skin of a subject. Various transdermal formulations that contain effective amounts of lauryl alcohol and isopropyl myristate to be administered to a subject are disclosed and described herein. Furthermore, methods of synergistically enhancing the penetration of a drug through the skin of a subject are also disclosed and described.

Any pharmaceutically acceptable transdermal formulation and method for administering a drug that does not interfere with the penetration enhancement described herein may be used for achieving the desired aspects of the present invention. The transdermal drug delivery system of the present invention may take a variety of well-known delivery formulations, including but not limited to, transdermal patches such as adhesive matrix patches, liquid reservoir system (LRS) patches, transmucosal patches or tablets, and topical formulations, such as creams, lotions, ointments, gels, pastes, mousses, aerosols, sprays, suppositories, etc.

When presented in the form of a transdermal patch, the transdermal drug delivery system of the present invention may include various structural components, as is known in the art. For example, in the case of an adhesive matrix patch, a distal backing is often laminated to a matrix polymer layer. Such a distal backing defines the side of the matrix patch that faces the environment, i.e., distal to the skin or mucosa. The backing layer functions to protect the matrix polymer layer and drug/enhancer composition and to provide an impenetrable layer that prevents loss of drug to the environment. Thus, the material chosen for the backing should be compatible with the polymer layer, drug, and enhancer, and should be minimally permeable to any components of the matrix patch. Advantageously, the backing can be opaque to protect components of the matrix patch from degradation from exposure to ultraviolet light. Furthermore, the backing should be capable of binding to and supporting the polymer layer, yet should be pliable enough to accommodate the movements of a person using the matrix patch.

Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. Additionally, the backing may include various foams, such as closed cell foams. Examples may include, without limitation, polyolefin foams, polyvinyl chloride foams, polyurethane foams, polyethylene foams, etc. In one aspect of the invention, the backing layer may have a thickness of about 0.0005 to 0.1 inch.

Further, a release liner may be temporarily provided upon the proximal side (side to adhere to the skin) of an adhesive layer. Such a liner provides many of the same functions as the backing layer, prior to adhesion of the patch to the skin. In use, the release liner is peeled from the adhesive layer just prior to application and discarded. The release liner can be made of the same materials as the backing layer, or other suitable films coated with an appropriate release surface.

In one general aspect, the transdermal drug delivery system of the present invention can comprise a pharmaceutically acceptable carrier intended to contain the drug and the combination of lauryl alcohol and isopropyl myristate. A number of pharmaceutically acceptable carriers are known to those of ordinary skill in the art and may be used in connection with the present invention.

Pharmaceutically acceptable carriers for use when the transdermal formulations of the present invention take the embodiment of an LRS patch may be any suitable viscous material known to those skilled in the art of transdermal drug delivery. Such carriers are typically a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. Such a viscous carrier may contain the combination of lauryl alcohol and isopropyl myristate and any drug to be transdermally delivered, as well as other optional components of the transdermal formulation.

Pharmaceutically acceptable carriers suitable for use when the present invention takes the embodiment of a transdermal matrix patch are also known to those of ordinary skill in the art. The present invention contemplates various structural types of transdermal matrix patches. For example, monolithic systems where the drug and enhancer are contained directly in a single pressure sensitive adhesive layer, as well as systems containing one or more polymeric reservoirs in addition to a pressure sensitive adhesive layer may be utilized. In aspects comprising systems having multiple layers/laminates, a rate controlling member may be included. Generally, a rate controlling member is located between a reservoir layer and the skin. In those aspects including a delivery layer and a reservoir layer, the rate controlling member may be adhered between a proximal side of the reservoir layer, and a distal side of the delivery layer. The rate controlling member is provided for the purpose of metering, or controlling, the rate at which drug and/or permeation enhancer migrates from the storage layer into the delivery layer. As noted herein, in one aspect of the present invention, various levels of permeation enhancement may be used to increase the delivery rate of the drug, and thus be used to vary other parameters, such as patch size, etc.

In one aspect, the pharmaceutically acceptable carrier used in a matrix patch can be a biocompatible polymer. Various general categories of biocompatible polymers are known, including, without limitation, rubbers; silicone polymers and copolymers; acrylic polymers and copolymers; and mixtures thereof. In one aspect, the biocompatible polymer can be a rubber, including natural and synthetic rubbers. One specific example of a useful rubber is a plasticized styrene-rubber block copolymer. In another aspect, the biocompatible polymer can include silicon polymers, polysiloxanes, and mixtures thereof. In yet another aspect, the biocompatible polymer can include acrylic polymers, polyacrylates, and mixtures thereof. In a further aspect, the biocompatible polymer can include vinyl acetates, ethylene-vinyl acetate copolymers, polyurethanes, plasticized polyether block amide copolymers, and mixtures thereof.

In one aspect, the biocompatible polymer of the pharmaceutically acceptable carrier can be suitable for long-term (e.g., greater than 1 day, may be about 3-4 days, or longer such as 7 days, or even 1-4 weeks) contact with the skin. In another aspect, the biocompatible polymer of the carrier is suitable for a short-term administration (e.g., for a few minutes to a few hours, less than or equal to 1 day). Such biocompatible polymers must be physically and chemically compatible with the combination of lauryl alcohol, isopropyl myristate, and any drug present, and with any carriers and/or vehicles or other additives incorporated into the formulation. In one aspect of the invention, the biocompatible polymers of the pharmaceutically acceptable carrier can include polymeric adhesives. Example of such adhesives can include without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof. In a further aspect of the invention, contact adhesives for use in the pharmaceutically acceptable carrier layer are acrylic adhesives, such as Duro-Tak®. 87-2888 adhesive (National Starch & Chemical Co., Bridgewater, N.J.); and polyisobutylene adhesives such as ARcare™. MA-24 (Adhesives Research, Glen Rock, Pa.) and ethylene vinyl acetate copolymer adhesives. In yet another aspect, gel-type or “hydrogel” adhesives are contemplated for use. See for example, U.S. Pat. No. 5,827,529 which is incorporated herein by reference. Those of ordinary skill in the art will appreciate that the specific type and amount of adhesive polymer used may be selected depending upon the desired specific characteristics of the final product. However, in one aspect, the amount of adhesive polymer in the adhesive matrix layer may be at least about 50% w/w of the adhesive layer. In another aspect, the amount may be at least about 60% w/w of the adhesive layer. In yet another aspect, the amount may be at least about 85% w/w of the adhesive layer. In a further aspect, the amount may be at least about 90% w/w of the adhesive layer. In an additional aspect, the amount may be from about 50% w/w to about 95% w/w of the adhesive layer.

Various pharmaceutically acceptable carriers which are known to those of ordinary skill in the art may be used when the transdermal formulations of the present invention take the embodiment of a topical formulation. In one aspect, the topical carrier can be an ointment including lauryl alcohol and isopropyl myristate. An ointment is a semisolid pharmaceutical preparation based on well known materials such as an oleaginous base, lanolin, emulsions, or water-soluble bases. Preparation of ointments is well known in the art such as described in Remington: The Science and Practice of Pharmacy 19^{th} ed. (1995), vol. 2, pp. 1585-1591, which is incorporated herein by reference. Such preparations often contain petrolatum or zinc oxide together with a drug. Oleaginous ointment bases suitable for use in the present invention include generally, but are not limited to, vegetable oils, animal fats, and semisolid hydrocarbons obtained from petroleum. Absorbent ointment bases of the present invention may contain little or no water and may include components such as, but not limited to, hydroxystearin sulfate, anhydrous lanolin and hydrophilic petrolatum. Emulsion ointment bases of the present invention are either water-in-oil (W/O) emulsions or oil-in-water (O/W) emulsions, and may include, but are not limited to, cetyl alcohol, glyceryl monostearate, lanolin, polyalkylsiloxanes, and stearic acid. Water-soluble ointment bases suitable for use in the present invention may be prepared from polyethylene glycols of varying molecular weight.

In another aspect of the present invention, the topical carrier can be a cream including lauryl alcohol and isopropyl myristate. Creams are a type of ointment which are viscous liquids or semisolid emulsions, either oil-in-water or water-in-oil, as is well known in the art. Cream bases may be soluble in water, and contain an oil phase, an emulsifier, an aqueous phase, and the active agent. In a detailed aspect of the present invention, the oil phase may be comprised of petrolatum and a fatty alcohol such as cetyl or stearyl alcohol. In another detailed aspect of the present invention, the aqueous phase may exceed the oil phase in volume, and may contain a humectant. In another detailed aspect of the present invention, the emulsifier in a cream formulation may be a nonionic, anionic, cationic or amphoteric surfactant.

In another aspect of the present invention, the topical carrier can be a lotion including lauryl alcohol and isopropyl myristate. A lotion is an ointment which may be a liquid or semi-liquid preparation in which solid particles, including the active agent, are present in a water or alcohol base. Lotions suitable for use in the present invention may be a suspension of solids or may be an oil-in-water emulsion. In another aspect of the present invention, lotions may also contain suspending agents which improve dispersions or other compounds which improve contact of the active agent with the skin, e.g., methylcellulose, sodium carboxymethylcellulose, or similar compounds.

In yet another aspect of the present invention, a topical carrier can be a paste including lauryl alcohol and isopropyl myristate. Pastes of the present invention are ointments in which there are significant amounts of solids which form a semisolid formulation in which the active agent is suspended in a suitable base. In a detailed aspect of the present invention, pastes may be formed of bases to produce fatty pastes or made from a single-phase aqueous gel. Fatty pastes suitable for use in the present invention may be formed of a base such as petrolatum, hydrophilic petrolatum or the like. Pastes made from single-phase aqueous gels suitable for use in the present invention may incorporate cellulose based polymers such as carboxymethylcellulose or the like as a base.

In another aspect of the present invention, a topical gel may be prepared. A gel prepared in accordance with the present invention may be a preparation of a colloid in which a disperse phase has combined with a continuous phase to produce a viscous product. The gelling agent may form submicroscopic crystalline particle groups that retain the solvent in the interstices. As will be appreciated by those working in art, gels are semisolid, suspension-type systems. Single-phase gels can contain organic macromolecules distributed substantially uniformly throughout a carrier liquid, which may be aqueous or non-aqueous and may contain an alcohol or oil.

In addition to containing the enhancer combination and a drug, the pharmaceutically acceptable carriers of the transdermal formulations recited herein, may include a number of other additives, such as diluents, excipients, emollients, plasticizers, solubilizers, skin irritation reducing agents, stabilizing compounds, or a mixture thereof. These types of components, as well as others not specifically recited, are well known in the art for inclusion in various transdermal formulations, and may be added as desired to the transdermal drug delivery system of the present invention in specific types and amounts in order to achieve a desired result. For example, polyvinylpyrrolidone can be utilized as a solubilizer in the pharmaceutically acceptable carrier at an amount from about 8% w/w to about 12% w/w of the transdermal formulation. In one aspect, polyvinylpyrrolidone can be present at an amount of about 10% of the transdermal formulation.

Furthermore, when the drug to be delivered is susceptible to acid catalyzed degradation, such as certain androgens and progestins, carriers that contain no acid functional groups, and that do not form any acid functional groups upon storage can be used in order to improve the stability of the formulation. One specific example of such a carrier is an ethylhexylacrylate polymer, as described in U.S. Pat. No. 5,780,050, which is incorporated by reference herein.

The present invention can be used to deliver a wide variety of drugs to a subject. The inventors have found that the synergistic combination of lauryl alcohol and isopropyl myristate may be particularly effective when used to deliver hormones. As such, in one aspect the hormone is a sex hormone. Various sex hormones including androgens, estrogens and progestins can be synergistically enhanced and delivered as disclosed herein.

Examples of specific androgens such as testosterone and related compounds include without limitation: testosterone, methyltestosterone, androstenedione, adrenosterone, dehydroepiandrosterone, oxymetholone, fluoxymesterone, methandrostenolone, testosterone, methyltestosterone, androstenedione, adrenosterone, dehydroepiandrosterone, oxymetholone, fluoxymesterone, methandrostenolone, testolactone, pregnenolone, 17α-methylnortestosterone, norethandrolone, dihydrotestosterone, danazol, oxymetholone, androsterone, nandrolone, stanozolol, ethylestrenol, oxandrolone, bolasterone and mesterolone, testosterone propionate, testosterone cypionate, testosterone phenylacetate, testosterone enanthate, testosterone acetate, testosterone buciclate, testosterone heptanoate, testosterone decanoate, testosterone caprate, testosterone isocaprate, and combinations thereof. These testosterone compounds can be present in subsaturated concentrations, or low concentrations. Examples of compositions comprising subsaturated testosterone are known in the art. See, for example, U.S. Pat. Nos. 5,164,190, and 5,152,997, which are incorporated herein by reference.

Examples of specific estrogens, such as estradiol and related compounds include without limitation: 17β-estradiol, 17α-estradiol, conjugated equine estrogen, esterified estrogen, micronized estradiol, sodium estrogen sulfate, ethinyl estradiol, estrone, tibolone, selective estrogen receptor modulator (SERM), phytoestrogen, and mixtures thereof.

Examples of specific progestins include, without limitation, progesterone, hydroxyprogesterone, megestrol acetate, dimethisterone, norgestrel, levonorgestrel, medroxyprogesterone acetate, desogestrel, norgestimate, ethynodiol diacetate, norethindrone, norethindrone acetate, norethynodrel and derivatives thereof.

The concentrations of hormones included in the transdermal formulations of the present invention can vary depending on the particular hormone ingredient, the particular carrier to be used, and the intended use of the formulation. It is well within the ability of one skilled in the art to formulate a proper hormone concentration for inclusion in the transdermal formulation. As such, any therapeutically effective amount of a hormone or drug is considered to be within the scope of the present invention. In one embodiment including testosterone for example, testosterone can be present in from about 0.5% w/w to about 15% w/w. In another embodiment, the testosterone can be present from about 1% w/w to about 10% w/w. In a further embodiment, the testosterone can be present from about 5% to about 12%. In yet another embodiment, the testosterone can be present from about 7% w/w to about 10% w/w.

As has been described herein, it has been discovered that a combination of lauryl alcohol and isopropyl myristate synergistically enhances the penetration of a drug through the skin of a subject. It is appreciated that variations in the level of synergism may occur between different transdermal formulations due to their disparate chemical make-up. For example, different drugs or carriers can exhibit different penetration enhancements when combined with the enhancer combinations of the present invention. As such, it is intended that any level of enhancement demonstrating synergism be within the scope of the present invention. More specifically, in one aspect the synergistically enhanced penetration can be from about 5% to about 150% greater than would be expected of an additive effect from using lauryl alcohol and isopropyl myristate. In another aspect, the synergistically enhanced penetration can be from about 5% to about 75% greater than would be expected. In yet another aspect, the synergistically enhanced penetration can be from about 10% to about 50% greater than would be expected. In a further aspect, the synergistically enhanced penetration can be less than or equal to about 5% greater than would be expected.

The amount of the enhancer combination included in a transdermal formulation can vary according to numerous factors, such as the desired level of enhancement for a particular drug, the potency of the drug, the duration of usage of the transdermal formulation, etc. Formulating the proper enhancer concentrations in a formulation required to attain a specific penetration enhancement effect can readily be determined by one skilled in the art. As such, any amount of the enhancer combination of the present invention that exhibits a synergistically enhanced effect is considered to be within the scope of the present invention. More specifically, in one aspect the transdermal formulation can include lauryl alcohol and isopropyl myristate that are each from about 0.5% w/w to about 20% w/w of the transdermal formulation. In another aspect, the lauryl alcohol and the isopropyl myristate can each be from about 1% w/w to about 10% w/w of the transdermal formulation. In another aspect, the lauryl alcohol and the isopropyl myristate each can be from about 22% w/w to about 78% w/w of the transdermal formulation. In yet another aspect, the lauryl alcohol and the isopropyl myristate can each be about 5% w/w of the transdermal formulation. In a further aspect, the lauryl alcohol and the isopropyl myristate can each be less than or equal to about 5% w/w of the transdermal formulation.

In addition to the concentration of the enhancer combination in the transdermal formulation, various ratios of lauryl alcohol to isopropyl myristate are contemplated and may be fine-tuned to achieve a desired level of penetration enhancement for a given transdermal formulation. That being stated, any ratio of lauryl alcohol to isopropyl myristate that achieves a synergistic result is considered to be within the scope of the present invention. In one aspect, the ratio can be from about 1:5 to about 5:1. In another aspect, the ratio can be from about 1:4 to about 4:1. In another aspect, the ratio can be from about 1:3 to about 3:1. In yet another aspect, the ratio can be from about 1:2 to about 2:1. In a more specific aspect, the ratio can be about 1:1. In yet another more specific aspect, the ratio can be about 4:1.

Various temporal orders of administering the components of the enhancer combination are possible, and any such order of administration that obtains a synergistic result is considered to be within the scope of the present invention. In one aspect, the lauryl alcohol and the isopropyl myristate can be administered concomitantly, either as a single composition or as separate compounds. Such concurrent administration is intended to include application of each of the compounds at essentially the same time. In such concurrent administration, the enhancer combination can be delivered concomitantly with, or separately from the drug. For the case of concomitant administration of the enhancer combination with the drug, the enhancer combination can be admixed with the drug or administered as separate compounds. Similarly, either of the lauryl alcohol or the isopropyl myristate can be admixed with the drug and the remaining component of the enhancer combination can be administered as a separate compound. In the case of separate administration of the enhancer combination with respect to the drug, the lauryl alcohol and isopropyl myristate can be administered prior to, following, or both prior to and following the administration of the drug.

In another aspect, the lauryl alcohol and the isopropyl myristate can be administered at disparate times from one another, as long as the disparate application still provides a synergistic penetration enhancement effect. As such, the lauryl alcohol can be administered either prior to or following application of the isopropyl myristate. Various temporal orders can be appreciated for application of the enhancers with respect to the drug. For example, the lauryl alcohol and isopropyl myristate can be administered prior to, following, or both prior to and following the administration of the drug. It is also contemplated that the drug can be administered concomitantly with one of the enhancer components.

The transdermal formulations of the present invention can be formulated to as sustained release formulations that administer therapeutically effective amounts of a drug over an extended period of time. As such, in one aspect, the sustained delivery period of the progestin may be for at least 7 days. In another aspect, the sustained delivery period may be at least 5 days. In a further aspect, the sustained delivery period may be at least 3 days. In another aspect, the sustained delivery period may be at least one day. In yet another aspect, the sustained delivery period may be less than one day. In a further aspect, the sustained delivery period may be from about 1 to about 4 weeks.

In addition to the methods and formulations for providing synergistically enhanced penetration of a drug through the skin of a subject, the present invention includes aspects directed towards kits for providing the synergistically enhanced penetration of a drug according to the methods disclosed herein. The kit can include a pharmaceutically acceptable carrier having a therapeutically effective amount of a drug admixed therein, an effective amount of lauryl alcohol, an effective amount of isopropyl myristate, and instructions describing a method of using the transdermal formulation, wherein the lauryl alcohol and the isopropyl myristate provide synergistically enhanced transdermal penetration of the drug. In one aspect, the lauryl alcohol and the isopropyl myristate can be combined as a single penetration enhancer composition, either separate from or admixed with the drug. In another aspect, the lauryl alcohol and the isopropyl myristate can be provided as separate compounds in the kit, either separate from or one compound admixed with the drug.

### EXAMPLES

The following examples of formulations providing synergistically enhanced drug penetration through the skin of a subject are provided to promote a more clear understanding of certain embodiments of the present invention, and are in no way meant as a limitation thereon.

### Example 1

Transdermal matrix systems containing lauryl alcohol (LA) and isopropyl myristate (IPM) can be made as follows: The solids content of an acrylic adhesive solution (Duro-Tak® 87-9301) are determined by placing small amounts into pre-weighed aluminum dishes which are then put in a convection oven (Model A4718-Q, Blue M) at 75° C. overnight. Following evaporation of the solvents, the weight of the dry adhesive is obtained and the solids content calculated as a ratio of the dry to wet weight.

An appropriate amount of polyvinylpyrrolidone K-12 (PVP) to yield 10% w/w in a dried film is weighed directly into a glass bottle with the minimum amount of absolute ethanol calculated to completely dissolve the PVP. A known quantity of the adhesive is weighed into the bottle based on previously determined solids content. An appropriate quantity of testosterone (TS) is added to the bottle to give about an 8.5% w/w TS concentration upon drying. LA is a solid at room temperature and can be melted by heating a small quantity held in a glass jar in a water bath prior to use. Appropriate amounts of LA and IPM are added to the bottles to give a desired composition having about 5% w/w of LA and 5% w/w of IPM upon drying. Each bottle is tightly capped, sealed with parafilm and rotated overnight during which time all ingredients dissolve to yield a clear solution.

An appropriate amount of the composition (about 10 g) is placed onto the high release side of a fluoropolymer coated 3 Mil thick polyester (PET) release liner and manually cast with a 10 mil gap casting knife. The cast is dried in a draft oven at 75° C. for 15 minutes. The film is left to cool for 10 minutes at room temperature. After drying, the cast is then laminated with a 2 mil (PET/EVA) laminate backing film. The cast and backing film can then be cut to provide a proper delivery dosage.

### Example 2

In vitro skin flux studies can be conducted using modified Franz diffusion cells. Heat separated human cadaver epidermal membranes can be used. Circular punches of 0.71 cm² are cut from the matrix patches of Example 1. The release liner is peeled and discarded and the matrix disc laminated onto the stratum corneum side of the epidermal membrane. The skin-matrix assembly is then sandwiched between the donor and receiver chambers of a diffusion cell and clamped in place with the epidermal side facing the receiver compartment. The receiver compartment is filled with 0.02% w/v sodium azide solution. The cells are then placed in a circulating water bath maintained at about 37° C.

At pre-selected time points, the entire contents of the receiver compartment are collected for drug quantitation. The receiver compartment is then re-filled with fresh receiver medium (0.02% w/v aqueous NaN₃). The interval flux and cumulative amount of drug permeating per unit area are calculated following HPLC analysis of the samples. The cumulative amount of drug (Qt, μg/cm²) permeating per unit area at any time t is given by:

$${Q}_{t} = \frac{{\sum }_{n = 0}^{t} ( {C}_{n} * V )}{A} \qquad ( 6 )$$

Where Cₙ is the concentration in μg/ml of the drug in the receiver medium at the corresponding sampling time, V is the volume of the receiver compartment (≈6.3 ml), and A is the diffusional area of the cell (0.64 cm²).

Mean flux and 24 hr cumulative permeation results are shown in Tables 1 and 2, respectively.

TABLE 1
Testosterone in 87-9301 Adhesive

| % Drug | % PVP | % LA | % IPM | Mean Flux | % Enhancement |
|---|---|---|---|---|---|
| 8.5% TS | 10 | 0 | 0 | 2.55 | Baseline |
| 8.5% TS | 10 | 5 | 0 | 3.37 | 32% |
| 8.5% TS | 10 | 0 | 5 | 3.03 | 19% |
| | | | | | *51% |
| 8.5% TS | 10 | 5 | 5 | 4.36 | 71% |
| | | | | | **39% |

*additive
**% increase

TABLE 2
Testosterone in 87-9301 Adhesive

| | | | % | 24 hr Cumulative | % |
|---|---|---|---|---|---|
| % Drug | % PVP | % LA | IPM | Permeation | Enhancement |
| 8.5% TS | 10 | 0 | 0 | 56.4 | Baseline |
| 8.5% TS | 10 | 5 | 0 | 80.8 | 43% |
| 8.5% TS | 10 | 0 | 5 | 72.8 | 29% |
| | | | | | *72% |
| 8.5% TS | 10 | 5 | 5 | 104.7 | 86% |
| | | | | | **19% |

*additive
**% increase

As can be seen from the data, there is a surprising synergistic increase in mean flux and permeation enhancement across an area of skin. The additive percentage noted in the tables represents the expected additive effect of the LA and IPM enhancers. The % increase noted in the tables represents the increase in enhancement above the additive percentage that is observed due to the actual effect of the combination of LA and IPM. It is this value that measures the percentage of synergism accomplished by the enhancer combination. The following are examples showing the permeation enhancement characteristics of LA and IPM in transdermal formulations containing TS and other hormones in various adhesives. Similar or greater levels of synergistic permeation enhancement are also observed in these cases.

### Example 3

Patches are made as in Example 1 with the exception that the adhesive is Duro-Tak® 87-900A. Patches are tested as described in Example 2. Mean flux and 24 hr cumulative permeation results are shown in Tables 3 and 4, respectively.

TABLE 3
Testosterone in 87-900A Adhesive

| % Drug | % PVP | % LA | % IPM | Mean Flux | % Enhancement |
|---|---|---|---|---|---|
| 8.5% TS | 10 | 0 | 0 | 2.68 | Baseline |
| 8.5% TS | 10 | 5 | 0 | 3.52 | 31% |
| 8.5% TS | 10 | 0 | 5 | 3.12 | 16% |
| | | | | | *47% |
| 8.5% TS | 10 | 5 | 5 | 4.53 | 69% |
| | | | | | **47% |

*additive
**% increase

TABLE 4
Testosterone in 87-900A Adhesive

| | | | % | 24 hr Cumulative | % |
|---|---|---|---|---|---|
| % Drug | % PVP | % LA | IPM | Permeation | Enhancement |
| 8.5% TS | 10 | 0 | 0 | 64.4 | Baseline |
| 8.5% TS | 10 | 5 | 0 | 84.6 | 31% |
| 8.5% TS | 10 | 0 | 5 | 75.1 | 17% |
| | | | | | *48% |
| 8.5% TS | 10 | 5 | 5 | 108.8 | 69% |
| | | | | | **44% |

*additive
**% increase

### Example 4

Patches are made as in Example 1 with the exception that the adhesive is silicone adhesive MD7-4502 (Dow Corning®) and the concentration of TS is 1%. Patches are tested as described in Example 2. Mean flux and 24 hr cumulative permeation results are shown in Tables 5 and 6, respectively.

TABLE 5
Testosterone in Silicone Adhesive MD7-4502

| % Drug | % PVP | % LA | % IPM | Mean Flux | % Enhancement |
|---|---|---|---|---|---|
| 1% TS | 10 | 0 | 0 | 1.23 | Baseline |
| 1% TS | 10 | 5 | 0 | 1.78 | 45% |
| 1% TS | 10 | 0 | 5 | 1.44 | 17% |
| | | | | | *62% |
| 1% TS | 10 | 5 | 5 | 2.13 | 73% |
| | | | | | **18% |

*additive
**% increase

TABLE 6
Testosterone in Silicone Adhesive MD7-4502

| | | | | 24 hr Cumulative | % |
|---|---|---|---|---|---|
| % Drug | % PVP | % LA | % IPM | Permeation | Enhancement |
| 1% TS | 10 | 0 | 0 | 29.5 | Baseline |
| 1% TS | 10 | 5 | 0 | 42.8 | 45% |
| 1% TS | 10 | 0 | 5 | 34.5 | 17% |
| | | | | | *62% |
| 1% TS | 10 | 5 | 5 | 51.7 | 75% |
| | | | | | **21% |

*additive
**% increase

### Example 5

Patches are made as in Example 1 with the exception that the hormone is estradiol (ES). Patches are tested as described in Example 2. Mean flux, 72 hr cumulative permeation, and day 3 result are shown in Tables 7, 8, and 9, respectively.

TABLE 7
Estradiol in 87-9301 Adhesive

| % Drug | % PVP | % LA | % IPM | Mean Flux | % Enhancement |
|---|---|---|---|---|---|
| 5% ES | 10 | 0 | 0 | 0.39 | Baseline |
| 5% ES | 10 | 5 | 0 | 0.67 | 72% |
| 5% ES | 10 | 0 | 5 | 0.46 | 18% |
| | | | | | *90% |
| 5% ES | 10 | 5 | 5 | 0.88 | 126% |
| | | | | | **40% |

*additive
**% increase

TABLE 8
Estradiol in 87-9301 Adhesive

| | | | % | 72 hr Cumulative | |
|---|---|---|---|---|---|
| % Drug | % PVP | % LA | IPM | Permeation | % Enhancement |
| 5% ES | 10 | 0 | 0 | 31.5 | Baseline |
| 5% ES | 10 | 5 | 0 | 52.6 | 67% |
| 5% ES | 10 | 0 | 5 | 37.2 | 18% |
| | | | | | *85% |
| 5% ES | 10 | 5 | 5 | 67.5 | 114% |
| | | | | | **34% |

*additive
**% increase

TABLE 9
Estradiol in 87-9301 Adhesive

| | | | % | Day 3 | % Enhancement From |
|---|---|---|---|---|---|
| % Drug | % PVP | % LA | IPM | Permeation | Day 3 Data*** |
| 5% ES | 10 | 0 | 0 | 8.7 | Baseline |
| 5% ES | 10 | 5 | 0 | 14.6 | 68% |
| 5% ES | 10 | 0 | 5 | 10.6 | 22% |
| | | | | | *90% |
| 5% ES | 10 | 5 | 5 | 19.5 | 124% |
| | | | | | **38% |

*additive
**% increase
***Based on permeation only from day 3.

### Example 6

Patches are made as in Example 1 with the exception that the hormone is norethindrone acetate (NEA). Patches are tested as described in Example 2. Mean flux, 168 hr cumulative permeation, and day 7 result are shown in Tables 10, 11, and 12, respectively.

TABLE 10
Norethindrone Acetate in 87-900A Adhesive

| % Drug | % PVP | % LA | % IPM | Mean Flux | % Enhancement |
|---|---|---|---|---|---|
| 10% NEA | 10 | 0 | 0 | 0.35 | Baseline |
| 10% NEA | 10 | 5 | 0 | 0.52 | 49% |
| 10% NEA | 10 | 0 | 5 | 0.42 | 20% |
| | | | | | *69% |
| 10% NEA | 10 | 5 | 5 | 0.68 | 94% |
| | | | | | **36% |

*additive
**% increase

TABLE 11
Norethindrone Acetate in 87-900A Adhesive

| | | | | 168 hr | |
|---|---|---|---|---|---|
| | | | | Cumulative | |
| % Drug | % PVP | % LA | % IPM | Permeation | % Enhancement |
| 10% NEA | 10 | 0 | 0 | 62.5 | Baseline |
| 10% NEA | 10 | 5 | 0 | 90.5 | 45% |
| 10% NEA | 10 | 0 | 5 | 73.3 | 17% |
| | | | | | *62% |
| 10% NEA | 10 | 5 | 5 | 119.0 | 90% |
| | | | | | **45% |

*additive
**% increase

TABLE 12
Norethindrone Acetate in 87-900A Adhesive

| | | | | | % Enhancement |
|---|---|---|---|---|---|
| | | | | Day 7 | From |
| % Drug | % PVP | % LA | % IPM | Permeation | Day 7 Data*** |
| 10% NEA | 10 | 0 | 0 | 7.5 | Baseline |
| 10% NEA | 10 | 5 | 0 | 11.4 | 52% |
| 10% NEA | 10 | 0 | 5 | 9.2 | 23% |
| | | | | | *75% |
| 10% NEA | 10 | 5 | 5 | 15.0 | 100% |
| | | | | | **33% |

*additive
**% increase
***Based on permeation only from day 7.

### Example 7

Patches are made as in Example 1 with the exception that the adhesive is Duro-Tak® 87-2516 and the hormone is norelgestromin (NG). Patches are tested as described in Example 2. Mean flux, 168 hr cumulative permeation, and day 7 result are shown in Tables 13, 14, and 15, respectively.

TABLE 13
Norelgestromin in 87-2516 Adhesive

| % Drug | % PVP | % LA | % IPM | Mean Flux | % Enhancement |
|---|---|---|---|---|---|
| 10% NG | 10 | 0 | 0 | 0.20 | Baseline |
| 10% NG | 10 | 5 | 0 | 0.26 | 30% |
| 10% NG | 10 | 0 | 5 | 0.22 | 10% |
| | | | | | *40% |
| 10% NG | 10 | 5 | 5 | 0.36 | 80% |
| | | | | | **100% |

*additive
**% increase

TABLE 14
Norelgestromin in 87-2516 Adhesive

| | | | | 168 hr | |
|---|---|---|---|---|---|
| | | | | Cumulative | |
| % Drug | % PVP | % LA | % IPM | Permeation | % Enhancement |
| 10% NG | 10 | 0 | 0 | 35.6 | Baseline |
| 10% NG | 10 | 5 | 0 | 44.5 | 25% |
| 10% NG | 10 | 0 | 5 | 38.6 | 8% |
| | | | | | *33% |
| 10% NG | 10 | 5 | 5 | 60.8 | 71% |
| | | | | | **115% |

*additive
**% increase

TABLE 15
Norelgestromin in 87-2516 Adhesive

| | | | | | % Enhancement |
|---|---|---|---|---|---|
| | | | | Day 7 | From |
| % Drug | % PVP | % LA | % IPM | Permeation | Day 7 Data*** |
| 10% NG | 10 | 0 | 0 | 4.2 | Baseline |
| 10% NG | 10 | 5 | 0 | 5.4 | 29% |
| 10% NG | 10 | 0 | 5 | 4.5 | 7% |
| | | | | | *36% |
| 10% NG | 10 | 5 | 5 | 7.7 | 83% |
| | | | | | **131% |

*additive
**% increase
***Based on permeation only from day 7.

As can be seen from the above-recited results from Examples 2-7, LA and IPM in combination provide synergistic enhancement above what would be expected of the additive effect of the enhancers. This synergistic effect was observed in all hormones tested.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of enhancing penetration of a drug through an area of skin, comprising:
administering a combination of lauryl alcohol and isopropyl myristate as a penetration enhancer to the area of skin to provide synergistically enhanced penetration of the drug.

2. The method of claim 1, wherein the synergistically enhanced penetration is from about 1% to about 150% greater than would be expected of an additive effect from using lauryl alcohol and isopropyl myristate.

3. The method of claim 2, wherein the synergistically enhanced penetration is from about 5% to about 75% greater than would be expected of an additive effect from using lauryl alcohol and isopropyl myristate.

4. The method of claim 3, wherein the synergistically enhanced penetration is from about 10% to about 50% greater than would be expected of an additive effect from using lauryl alcohol and isopropyl myristate.

5. The method of claim 1, wherein the penetration enhancer includes lauryl alcohol and isopropyl myristate in a ratio of from about 1:5 to about 5:1.

6. The method of claim 5, wherein the penetration enhancer includes lauryl alcohol and isopropyl myristate in a ratio of from about 1:4 to about 4:1.

7. The method of claim 6, wherein the penetration enhancer includes lauryl alcohol and isopropyl myristate in a ratio of from about 1:3 to about 3:1.

8. The method of claim 7, wherein the penetration enhancer includes lauryl alcohol and isopropyl myristate in a ratio of from about 1:2 to about 2:1.

9. The method of claim 1, wherein the penetration enhancer includes lauryl alcohol and isopropyl myristate in a ratio of about 1:1.

10. The method of claim 1, wherein the penetration enhancer includes lauryl alcohol and isopropyl myristate in a ratio of about 4:1.

11. The method of claim 1, wherein the drug is a hormone.

12. The method of claim 11, wherein the hormone is a sex hormone.

13. The method of claim 12, wherein the sex hormone is a member selected from the group consisting of testosterone, norethindrone, norethindrone acetate, estradiol, ethinyl estradiol, norelgestromin, and mixtures, salts, isomers, or analogues thereof.

14. The method of claim 13, wherein the sex hormone is testosterone.

15. The method of claim 1, wherein the lauryl alcohol is administered either prior to, concurrently with, or following the isopropyl myristate.

16. The method of claim 15, wherein the lauryl alcohol is administered concurrently with the isopropyl myristate.

17. The method of claim 1, wherein the combination of lauryl alcohol and isopropyl myristate is administered either prior to, concurrently with, or following the drug.

18. The method of claim 17, wherein the combination of lauryl alcohol and isopropyl myristate is administered concurrently with the drug.

19. The method of claim 1, wherein the combination of lauryl alcohol and isopropyl myristate is administered both before and after the drug.

20. The method of claim 1, wherein the combination of lauryl alcohol and isopropyl myristate is administered as a single enhancer composition.

21. A penetration enhancer composition for use as recited in claim 20, comprising:
a combination of lauryl alcohol and isopropyl myristate, wherein the combination provides synergistically enhanced penetration of a drug through the skin.

22. A transdermal formulation, comprising:
a pharmaceutically acceptable carrier;
a therapeutically effective amount of a drug; and
a penetration enhancer composition as recited in claim 21, wherein the penetration enhancer composition provides synergistically enhanced transdermal penetration of the drug.

23. The transdermal formulation of claim 22, wherein the drug is a hormone.

24. The transdermal formulation of claim 23, wherein the hormone is a sex hormone.

25. The transdermal formulation of claim 24, wherein the sex hormone is a member selected from the group consisting of testosterone, norethindrone, norethindrone acetate, estradiol, ethinyl estradiol, norelgestromin, and mixtures, salts, isomers, or analogues thereof.

26. The transdermal formulation of claim 25, wherein the sex hormone is testosterone.

27. The transdermal formulation of claim 22, wherein the lauryl alcohol and the isopropyl myristate are each from about 0.5% w/w to about 20% w/w of the transdermal formulation.

28. The transdermal formulation of claim 27, wherein the lauryl alcohol and the isopropyl myristate are each from about 1% w/w to about 10% w/w of the transdermal formulation.

29. The transdermal formulation of claim 28, wherein the lauryl alcohol and the isopropyl myristate are each from about 2.5% w/w to about 7.5% w/w of the transdermal formulation.

30. The transdermal formulation of claim 29, wherein the lauryl alcohol and the isopropyl myristate are each about 5% w/w of the transdermal formulation.

31. The transdermal formulation of claim 22, wherein the lauryl alcohol and the isopropyl myristate are each less than or equal to about 5% w/w of the transdermal formulation.

32. The transdermal formulation of claim 22, wherein the pharmaceutically acceptable carrier is a biocompatible polymer.

33. The transdermal formulation of claim 32, wherein the biocompatible polymer is a member selected from the group consisting of: rubbers; silicone polymers and copolymers; acrylic polymers and copolymers; and mixtures thereof.

34. The transdermal formulation of claim 33, wherein the biocompatible polymer is a rubber selected from the group consisting of: natural and synthetic rubbers, plasticized styrene-rubber block copolymers, and mixtures thereof.

35. The transdermal formulation of claim 33, wherein the biocompatible polymer is a member selected from the group consisting of: silicone polymers, polysiloxanes, and mixtures thereof.

36. The transdermal formulation of claim 33, wherein the biocompatible polymer is a member selected from the group consisting of: acrylic polymers, polyacrylates, and mixtures thereof.

37. The transdermal formulation of claim 32, wherein the biocompatible polymer is a member selected from the group consisting of: vinyl acetates, ethylene-vinyl acetate copolymers, polyurethanes, plasticized polyether block amide copolymers, and mixtures thereof.

38. The transdermal formulation of claim 22, wherein the pharmaceutically acceptable carrier comprises a viscous material suitable for use as a liquid reservoir.

39. The transdermal formulation of claim 38, wherein the viscous material forms a gel.

40. The transdermal formulation of claim 22, further comprising a member selected from the group consisting of: diluents, excipients, emollients, plasticizers, skin irritation reducing agents, stabilizing compounds, or mixtures thereof.

41. The transdermal formulation of claim 22, wherein the formulation is a transdermal patch.

42. The transdermal formulation of claim 41, wherein the transdermal patch is a transdermal matrix patch.

43. The transdermal formulation of claim 41, wherein the transdermal patch is a liquid reservoir patch.

44. The transdermal formulation of claim 22, wherein the formulation is a topical formulation.

45. The transdermal formulation of claim 44, wherein the topical formulation is a member selected from the group consisting of creams, lotions, ointments, gels, pastes, mousses, aerosols, sprays, waxes, balms, suppositories, and mixtures or combinations thereof.

46. A method of transdermally delivering a drug with enhanced penetration through an area of skin on a subject, comprising:
administering to the area of skin, a transdermal formulation as recited in claim 22.

47. A kit for administering a transdermal formulation having a drug with enhanced penetration through an area of skin on a subject, comprising:
a pharmaceutically acceptable carrier having a therapeutically effective amount of a drug admixed therein;
an effective amount of lauryl alcohol;
an effective amount of isopropyl myristate;
instructions describing a method of using the transdermal formulation, wherein the lauryl alcohol and the isopropyl myristate provide synergistically enhanced transdermal penetration of the drug.

48. The kit of claim 47, wherein the lauryl alcohol and the isopropyl myristate are combined as a single penetration enhancer composition.

49. A method of enhancing transdermal penetration of a drug through an area of skin on a subject, comprising:
administering to the area of skin, a transdermal formulation including;
a pressure sensitive acrylic polymer in an amount of from about 55% w/w to about 85% w/w of the transdermal formulation;
testosterone in an amount of from about 5% w/w to about 12% w/w of the transdermal formulation;
polyvinylpyrrolidone in an amount of from about 8% w/w to about 12% w/w of the transdermal formulation; and
a penetration enhancer composition including a combination of from about 2% w/w to about 8% w/w of lauryl alcohol and from about 2% w/w to about 8% w/w of isopropyl myristate, wherein the combination provides synergistically enhanced penetration of a drug through an area of skin, the synergistically enhanced penetration being from about 10% to about 50% greater than would be expected of an additive effect from using lauryl alcohol and isopropyl myristate.

50. A transdermal formulation having enhanced penetration of a drug, comprising:
a pressure sensitive acrylic polymer in an amount of from about 55% w/w to about 85% w/w of the transdermal formulation;
testosterone in an amount of from about 5% w/w to about 12% w/w of the transdermal formulation;
a solubilizer in an amount of from about 8% w/w to about 12% w/w of the transdermal formulation; and
a penetration enhancer composition including:
lauryl alcohol in an amount of from about 2% w/w to about 8% w/w of the transdermal formulation; and
isopropyl myristate in an amount of from about 2% w/w to about 8% w/w of the transdermal formulation, wherein the penetration enhancer composition provides synergistically enhanced penetration of the testosterone through an area of skin of a subject.

51. The formulation of claim 50, wherein the pressure sensitive acrylic polymer is from about 70% to about 73% w/w of the transdermal formulation.

52. The formulation of claim 50, wherein the testosterone is from about 7% w/w to about 10% of the transdermal formulation.

53. The formulation of claim 50, wherein the solubilizer is polyvinylpyrrolidone.

54. The formulation of claim 53, wherein the polyvinylpyrrolidone is about 10% w/w of the transdermal formulation.

55. The formulation of claim 50, wherein the lauryl alcohol is about 5% w/w of the transdermal formulation.

56. The formulation of claim 50, wherein the isopropyl myristate is about 5% w/w of the transdermal formulation.

57. A transdermal formulation having enhanced penetration of a drug, comprising:
a pressure sensitive acrylic polymer in an amount of from about 55% w/w to about 85% w/w of the transdermal formulation;
norethindrone acetate in an amount of from about 5% w/w to about 15% w/w of the transdermal formulation;
a solubilizer in an amount of from about 8% w/w to about 12% w/w of the transdermal formulation; and
a penetration enhancer composition including:
lauryl alcohol in an amount of from about 2% w/w to about 8% w/w of the transdermal formulation; and
isopropyl myristate in an amount of from about 2% w/w to about 8% w/w of the transdermal formulation, wherein the penetration enhancer composition provides synergistically enhanced penetration of the testosterone through an area of skin of a subject.

58. The formulation of claim 57, wherein the pressure sensitive acrylic polymer is from about 68% to about 72% w/w of the transdermal formulation.

59. The formulation of claim 57, wherein the norethindrone acetate is from about 8% w/w to about 12% w/w of the transdermal formulation.

60. The formulation of claim 57, wherein the solubilizer is polyvinylpyrrolidone.

61. The formulation of claim 60, wherein the polyvinylpyrrolidone is about 10% w/w of the transdermal formulation.

62. The formulation of claim 57, wherein the lauryl alcohol is about 5% w/w of the transdermal formulation.

63. The formulation of claim 57, wherein the isopropyl myristate is about 5% w/w of the transdermal formulation.
